(19) United States
(12) Patent Application Publication
(10) Pub. No.: US 2022/0273297 A1
(43) Pub. Date: Sep. 1, 2022

(54) Surgical Suture

(71) Applicant: Chandrashekar Balachandran, Killara (AU)

(72) Inventor: Chandrashekar Balachandran, Killara (AU)

(21) Appl. No.: 17/630,821

(22) PCT Filed: Jul. 30, 2020
(86) PCT No.: PCT/AU2020/050785

§ 371 Date: Jan. 27, 2022

(30) Foreign Application Priority Data
Jul. 30, 2019 (AU) 2019902720

(51) Int. Cl. A61B 17/06; A61B 17/04

(52) U.S. Cl. CPC A61B 17/06166; A61B 17/06066; A61B 17/0401; A61B 2017/0619

(57) ABSTRACT

A surgical suture comprising an elongate body having a proximal head for anchoring the suture; at least part of the elongate body being deformable to create a distal head, the surgical suture suitable to secure tissue between the proximal head and the distal head. There is also provided a method for inserting the surgical suture described above in order to close an incision, and a surgical suture system.

The present invention relates to surgical sutures used for wound closures, particularly for interrupted suturing.

### BACKGROUND

Sutures are designed to achieve closure of wounds/incisions. By way of example, corneal transplantation surgery sutures not only have to close the gap between the host and recipient cornea, they also have to apply equal tension in all directions. Anterior lamellar keratoplasty and penetrating keratoplasty involve at least 12-16 interrupted or 12-16 bite continuous sutures or a combination of both (see Prior Art FIGS. 1 and 2). The donor cornea at the time of suturing is often swollen and therefore it is difficult to control or predict the tension at the time of suturing and not until after surgery when the corneal swelling has resolved. At this stage If excessive tension is present in one or a few sutures then the graft can be pulled excessively in one direction leading to profound astigmatism and reduced vision. If the tension is not enough, then as the swelling disappears the suture will be found to be loose and the incision can gape.

The final astigmatism following closure of corneal incision(s) is a vector sum of the tension in each of the sutures. If a continuous suture is placed, the astigmatism can potentially be addressed by adjusting the suture tension toward the loose segments in the early postoperative period. However, running sutures can break during surgery or during adjustment in the postoperative period as they are very thin (10-0 Nylon 20 micron diameter). A broken suture would compromise incision integrity and would require either an immediate return to surgery for repeat suturing or the surgeon may have anticipated this possibility and placed a second running suture or interrupted sutures which can maintain wound integrity. These remaining sutures being integral to incision integrity would not be adjusted and therefore the astigmatism could not be adjusted.

Interrupted sutures are more popular. If a suture breaks there are other sutures (often 14-16) that can maintain integrity, there is no risk of urgent return to surgery. Currently an interrupted suture loops around the incision going to 90% depth and coming out superficially. The knot is tied and buried as for continuous suture into the donor cornea to minimise vascularisation and rejection.

Unlike continuous sutures interrupted sutures cannot be tightened or adjusted, they can only be removed. If the interrupted sutures are all equally tight then the tension is uniform and no astigmatism will be induced. However, usually one or more sutures are tighter than other sutures and this results in a vector of tension which causes astigmatism. The surgeon cannot always know if one or more sutures are too tight. It is only on removal of the suture that its effect can be gauged. If there was no change in astigmatism, then the suture which was providing wound integrity would have been needlessly removed. The surgeon now has to remove further sutures to correct the unchanged astigmatism. If all the sutures are removed and there is still a large amount of astigmatism, then there are no further options except either to consider the graft a failure or attempt other operations such as wedge excision and re-suturing. Overall the result can be poor astigmatism control and poor vision which is considered graft failure.

Currently non-absorbable sutures are used in corneal transplantation as they last longer than 60 days. A number of non-absorbable sutures are available such as, silk, polypropylene (Prolene), braided polyesters (Mersilene, Ethibond), and polybutester (Novafil™). The monofilament nylon is the most popular in corneal transplantation as, it is chemically inert, has a high tensile strength, high elasticity and low tissue reactivity. Unfortunately, it has significant memory and is not very pliable. Some effort has been made to reduce the disadvantages by soaking it in alcohol.

Polypropylene (Prolene) is similar to nylon in its tensile strength and low tissue reactivity. It is a smooth monofilament which is too smooth for knot security and needs extra throws. It has high plasticity and so can accommodate for wound swelling and de-swelling.

Polybutester is a thermoplastic copolymer of poly-glycol terephthate and polybutylene terephthate. It is the newest of the synthetic non-absorbable sutures and goes by the trade name Novafil™. Polybutester has greater strength and stiffness and elasticity than nylon or polypropylene.

### SUMMARY OF THE INVENTION

Embodiments of the invention provide a surgical suture suitable for insertion into tissue, the surgical suture has an elongated body portion for insertion into tissue and to extend at least partially out of the tissue and a proximal head portion to provide an anchor point to limit insertion of the suture into the tissue, wherein the length of the suture is defined by deforming at least part of the elongated body portion extending out of the tissue to create a distal head, to secure the tissue between the proximal head and the distal head.

In a broad concept, the invention provides a surgical suture comprising an elongate body having a proximal head for anchoring the suture; at least part of the elongate body being deformable to create a distal head, the surgical suture suitable to secure tissue between the proximal head and the distal head.

The present suture and method of using the suture provide a reliable and accurate means of fine adjustment of suture length, which depending on the suture's intended application, translates into accurate correction and adjustment of wounds. There is less room for human error in using the sutures as the process generally requires less manipulation of sutures than known techniques. The need for tying knots is obviated.

Preferably, the elongate body is deformable by at least one of: melting, polymerisation, or moulding with a solvent, and in a more preferable embodiment the elongate body is deformable when heated to the melting point of the material from which the elongate body is made.

The elongate body is preferably characterised by being suitable for insertion through and protruding at least partly out of tissue, the distal head being created from part, being a tip or a distal end, of the elongate body protruding out of the tissue after insertion. Accordingly, in one embodiment the suture will have suitable stiffness such that force applied to one head is transferred along the elongate body portion to the other head. This assists in pushing the elongate body out of the tissue, and specifically the tip or exposed distal end of the elongate body, as opposed to pulling the tip or distal end out of the tissue. However, the suture may be suitable to be either pushed, pulled or both. Where the suture requires a stiffness, it is considered that a suitable stiffness for the material of the suture could be a stiffness of 2.5×10⁹ Pa or greater. In a particular embodiment the stiffness could be 3.4×10⁹ Pa or greater.

The length of the suture is defined by the length of the elongate body portion between the proximal head and the distal head. In a preferred embodiment, the length of the suture is adjustable after creating the distal head, and namely after implantation. Such adjustment may be achieved by the elongate body portion being made of a deformable material that can be repeatedly cut and deformed to create a new distal head or a new proximal head.

In embodiments the length of the suture can be adjusted after insertion. The length can be standardised for specific graft sizes to achieve equal tension around the graft edge and minor adjustment after surgery.

In a preferred embodiment, the proximal head is integrally preformed with the elongate body portion. The proximal head can be a flanged head having a diameter that is at least twice the diameter of the elongate body, and even three times the diameter of the elongate body. The proximal head may be tapered out from elongate body to an outer flange perimeter at an angle of approximately 45° to 90°. This assists in the proximal head sitting upright and correctly on the tissue's surface.

A needle of some form may be required for the suture. In one embodiment a needle is attached to a distal end of the elongate body of the suture. In another embodiment, the elongate body itself is formed with a preformed sharp tip at a distal end of the elongate body to act as a needle.

The suture may have length markings along the elongate body portion to assist in visually identifying correct suture length and changes in adjustments, including shortening the suture length.

The present invention also provides a method for inserting the surgical suture described above in order to close an incision, the method comprising the steps of: inserting the elongate body portion into a first surface of tissue on a first side of an incision, across the incision and through the tissue to protrude out of the tissue on a second side of an incision until the proximal end anchors the surgical suture at the first surface of the tissue to prevent further insertion; deforming an exposed part of the elongated body portion protruding out of the tissue to create a distal head, the surgical suture closing the incision by securing the tissue between the proximal head and the distal head.

The method is preferably suitable for both initial implantation and subsequent adjusting of the length of the suture. Adjustment may be achieved by removing at least one of the proximal head or distal head to create a new exposed tip of the elongate body and deforming the tip to create a new head portion, resulting in the elongate body having a shorter length.

Deforming the exposed part or tip of the elongate body may be by heating the exposed part or tip and applying a pressing force to swage the exposed part or tip thereby creating a distal head. More specifically, deformation may be achieved by grasping the exposed part or tip using forceps having a flanged head and pressing down onto the exposed part or tip against the flanged forceps head using a heated plate to deform the exposed part or tip into a head. The forceps and heated plate can be part of a deformation device that uses heat to deform the elongate body.

There is further provided a surgical suture system comprising: a suture as described above, and a deformation device comprising a cautery mechanism and forceps for holding the elongate body out of the tissue, the cautery mechanism suitable for melting a section of the elongate body portion to create a head portion. The cautery mechanism may have a flat heated base plate or curved heated base plate. The base plate will be suitable for applying pressure to and melting the elongate body to create the head portion.

Furthermore, the forceps of the deformation device may comprise semi-elliptical grasping parts having a central opening through which the elongate body extends and is grasped, and the grasping parts are flanged so as to, in use, shield the tissue from heat generated by the cautery mechanism.

### BRIEF DESCRIPTION OF THE DRAWINGS

In order that the invention be more clearly understood and put into practical effect, reference will now be made to preferred embodiments of an assembly in accordance with the present invention. The ensuing description is given by way of non-limitative example only and is with reference to the accompanying drawings, wherein:

FIG. 1 illustrates a prior art example of an interrupted suture in a corneal transplant;

FIG. 2 illustrates a prior art example of a continuous suture in a corneal transplant;

FIGS. 3a and 3b illustrates examples of a grafted cornea using multiple sutures in accordance with the present invention;

FIG. 4 illustrates a suture in accordance with the present invention;

FIGS. 5a-d illustrates four steps in the process for inserting the suture into a corneal transplant, in accordance with an embodiment of the present invention;

FIGS. 6a-6c illustrates the process for adjusting the length of the suture after formation, in accordance with an embodiment of the present invention.

FIG. 7 illustrates a cautery and forceps mechanism used to deform the suture in an embodiment;

FIG. 8 illustrates sutures applied with markings; and

FIG. 9 illustrates a further embodiment of a suture according to the invention.

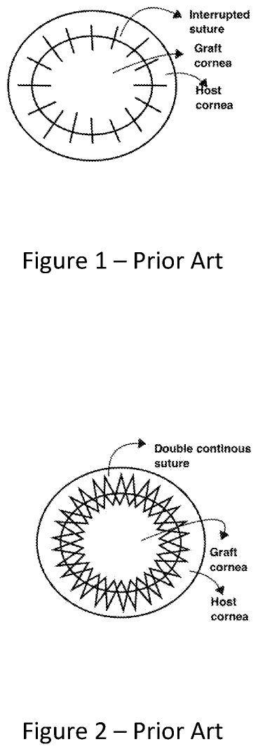

### DETAILED DESCRIPTION

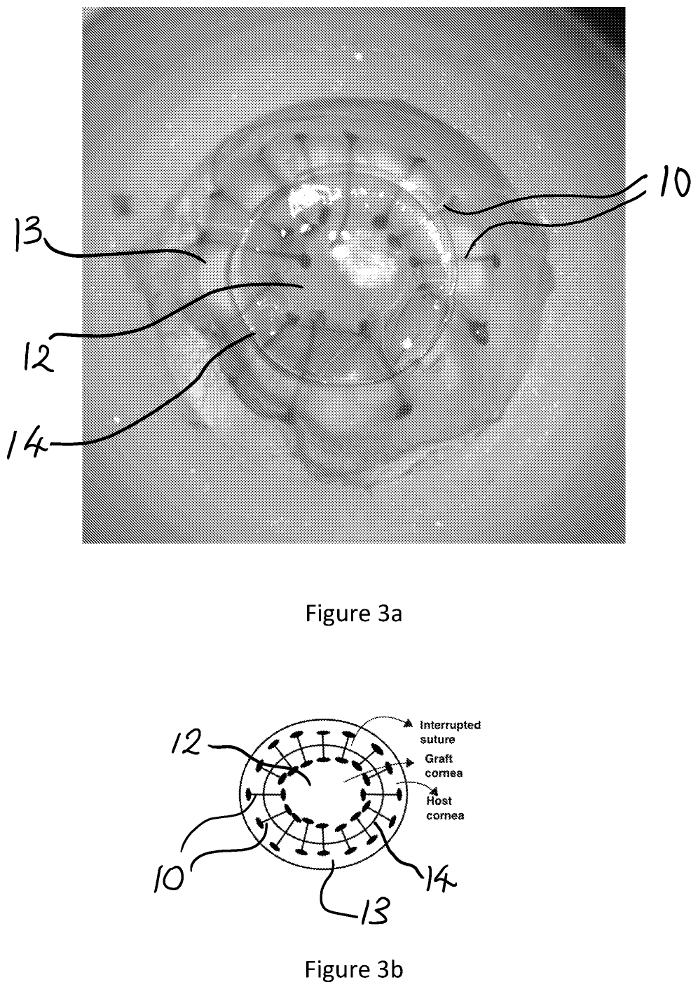

A surgical suture and a method of using the surgical suture is described herein with reference to the drawings. The present embodiments are described by reference to grafting a cornea transplant using the surgical sutures. FIGS. 3a and 3b illustrate, pictorially and schematically, a graft cornea 12 sutured onto a host cornea 13 using sutures 10 in accordance with the present invention. The sutures 10 are inserted to stitch together the graft and host corneas and follow an annular path at the incision. The number of stitches will vary and is dependent on the tensile strength of the suture material as well as the final astigmatism to be achieved in the eye.

While the description of the sutures and method of using the sutures refers herein specifically to the application of ophthalmic surgery such as corneal transplantation, it is understood that other uses of the surgical suture and accompanying method are also foreseeable. The surgical suture is suitable for use particularly where interrupted suturing is appropriate. Some non-limiting examples of such uses are in cosmetic surgery were a reproducible amount of tension is desired. For example in eyelid tightening procedures were tension of the lid is critical.

Furthermore, while in a preferred embodiment the suture 10 would be used for interrupted suturing, it is foreseeable that the concept of the present suture could also be applied to continuous suturing with minor modifications. For example, a longer suture would be required to enable multiple bites in tissue. Foreseeably, one-way barbs provided along the length of the suture would deter the suture slipping back.

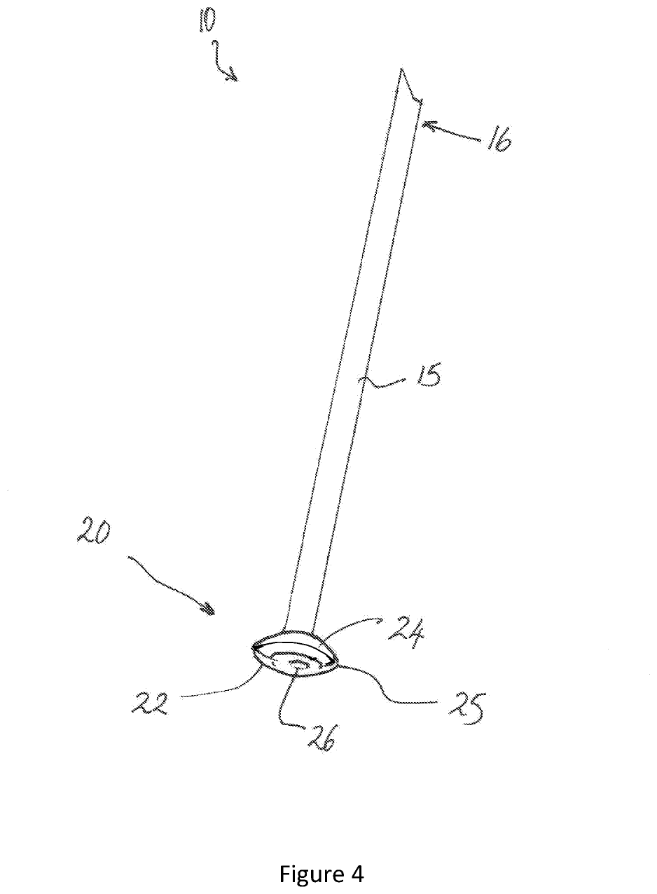

An embodiment of the inventive surgical suture is illustrated more closely in FIG. 4. The suture 10 includes an elongate body portion in the form of a suture shaft 15, and a proximal head 20, or cap, that is preformed to act as a first anchor at one end. At least a part of the shaft 15, and particularly a part at the distal end 16 of the shaft 15, is deformable so that a distal head 17 can be created after insertion through tissue to form a second anchor thereby securing tissue between the proximal head 20 and the distal head 17.

A deformable shaft not only allows a suture to be anchored when it is first inserted through tissue, but also allows for the suture length to be adjusted postoperatively, for example, to adjust astigmatism if the cornea is not appropriately tensioned all around, or if swelling reduces and the sutures become loose and need to be tightened.

Deformation of the shaft to create the distal head 17 may be by way of melting, polymerisation or moulding the suture material with a solvent. In the process described herein deformation is caused by heat and namely melting the distal end of the suture, specifically by using a diathermy machine, to mould or deform the tip into an anchoring head. However, the suture material may be moulded by means other than heat such as polymerisation or moulding with a solvent.

An advantage of the suture 10 is that it allows controlled tensioning and accurate suturing of tissue, which in turn leads to better surgical results.

Furthermore, the present invention provides a more effective technique of using sutures than used previously in that it allows adjustability over the course of healing so that the interrupted sutures can be tightened and adjusted independently. Tightening in particular is useful as the wound integrity will be enhanced. Sutures are tightened by adjusting the length of the suture. A controlled amount of tightening will allow some predictability to astigmatism management and it can be done in the clinic where immediate topography can be performed to titrate the process.

Sutures can also be removed independently. Alternatively, the sutures could be absorbable. However, it is envisaged that the sutures could remain in the eye indefinitely and without dissolving so as to maintain the desired degree of astigmatism through tension held by the sutures.

The present design of the surgical suture 10 can be described as operating like a cuff link, albeit a length adjustable cuff link. The suture material used is a material capable of melting within a workable temperature range of a diathermy machine yet not be too high so as to burn underlying tissue. A melting range of 40° C. to 300° C. would be suitable, where higher temperatures may require tissue screening. Thermoplastics have a wide range of melting points, some melting as low as 40° C. Examples of suitable thermoplastic suture materials include polybutester, with melting temperatures of 200-300° C. or polypropylene with a melting temperature close to 160° C.

Both the proximal and distal heads are formed using similar processes, only that the proximal head is preformed (e.g. in a factory setting) before use and the distal head is formed immediately after insertion into tissue.

After insertion, the suture's distal end 16 is melted to create a rounded or flat anchoring distal head 17. The preformed proximal head 20 will have a preformed flanged portion that anchors the suture from one end. The preformed head 20, as shown in FIG. 4, has a curved convex outer surface 22 over which the epithelium can grow over during healing. The suture 10 shown in FIG. 4 has not yet been attached with a needle. The outer surface 22 includes a small central depression 26 that can be used to centre a counter pressure instrument 23, as discussed in more detail below. The underside 24 of the proximal head 20 has an angle of between approximately 45-90° leading to the suture shaft 15 so that the head 20 sits substantially perpendicular to the corneal surface with respect to the cornea. The proximal head is 2-3 times greater than the diameter of the shaft 15 of the suture and its thickness tapers outwardly to an outer flange 25 of proximal head 20, where the flange has a thickness of 50 um (microns) or less. This small thickness will allow epithelium to grow over the head. The head is about 450 um in diameter.

The diameter of the suture shaft will be larger than a 10-0 nylon (0.020-0.029 mm), otherwise it is likely that deep placement will lead to superficial incision gape and superficial placement will lead to deeper incision gape. A larger suture such as a 5-0 (0.100-0.149 mm) will give a larger area over which to exert tension.

Each suture is provided with a separate needle 28. Accordingly, each of the 14-16 bites illustrated in FIGS. 3a and 3b were inserted using separate sutures 10 with needles 28. The suture may have a needle attached at one end by crimping a needle onto the suture or by having a preformed sharp tip at the distal end 16 with or without an arrow head. After insertion through tissue and before deformation of the distal end, the needle is cut away or otherwise melted or deformed itself.

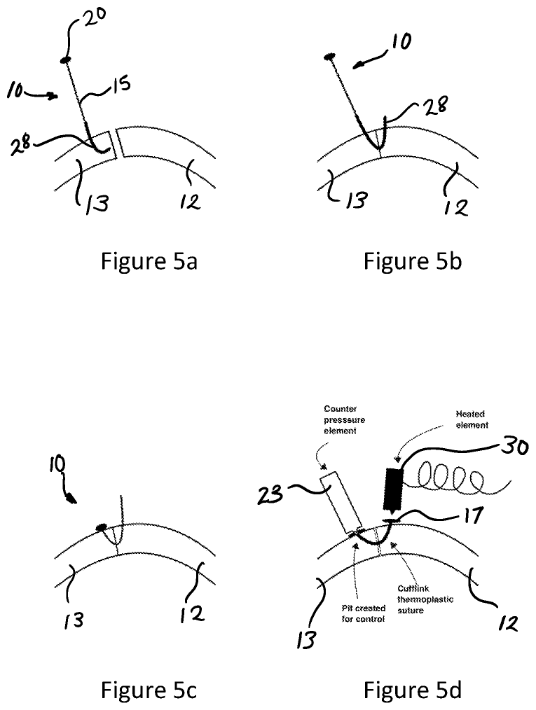

FIGS. 5a to 5d illustrate four steps in the process of using the suture 10 to stitch a circular graft cornea 12 into a corresponding hole cut from a cornea host 13. FIG. 5a shows the needle 28 is being inserted a finite distance of about 1 mm from the incision edge of the graft 12 to a depth of 60-80% of the depth of the epithelium. The needle is then passed across the incision, as shown in FIG. 5b, to the same depth to emerge out of the tissue on the receipt/host epithelium about 1 mm behind the incision. This step is shown in FIG. 5c. By controlling the incision distance, and the depth and length of the suture, the tension and therefore the astigmatism can be standardised.

The suture is pulled through until the preformed proximal head 20 abuts the cornea. Once the needle has passed through the cornea the suture is cut near the needle 28 to remove the needle, and the cut distal end is melted to create the second capped ending in the form of the distal head 17. If a non-needle perforation approach is used using a sharpened suture tip instead then the tip or the arrow head can be melted. This process has the added advantage of being quick and standard across the cornea. As shown in FIG. 5d, a counter pressure instrument 23 can be used to apply counter pressure to the preformed proximal head 20 to assist in pushing the distal end out of the tissue and allowing the distal end to be melted to form the distal head 17. Intraoperatively if the incision is not tight enough then the central convex part of the outer surface 22 of the proximal head 20 can be pushed into the cornea with the instrument 23 causing the peripheral distal tip to protrude, which will then be melted again to reduce the shaft length and tighten the incision closure.

The shaft length is taken to be the suture distance between the proximal and distal heads. The sutures can vary in length before use depending on application. In the current application of corneal transplantation the sutures, before use and as interrupted sutures, will be in the order of 20 mm in length. The suture shaft would generally be circular in cross-section but could alternatively have varying cross-sectional shapes including oval, rectangular, polygonal or have a flattened profile.

The suture material should have stress-strain characteristics that will enable force applied at one head to be transferred through the shaft to the opposite head. This stiffness is unlikely to result in a suture having the flexibility required to place a knot. This stiffness contributes to the profile of the suture resembling a cufflink-like profile. Furthermore, the suture material needs to have sufficient tensile strength to hold the incision wound closed and avoid leakage. By way of benchmark, closed wounds need to withstand 30-40 mmHg of pressure.

Polypropylene (prolene) sutures demonstrate good tensile strength and stiffness. A 5-0 or 6-0 suture or smaller would be appropriate for ophthalmic surgery as it would not be too large for the corneal thickness. The average cornea at the periphery is about 700 um, a 5-0 prolene would be about 100 um and have a tensile strength of 7N and a stiffness (Young's Modulus) of 3.4×10⁹ Pa or greater, and therefore a low elasticity. Compared to a Nylon suture having a lower tensile strength and a significantly lower stiffness, prolene provides a suitable material for present application of the suture. Similarly polybutester shows good tensile strength and stiffness. It is thought that a stiffness of 2.5×10⁹ Pa or greater would be suitable as a material for the suture, in the embodiment where stiffness is desired to assist in pushing the suture through the tissue. Of course, the alternative is to pull a less stiff suture from the distal end and deform the distal end while pulling on the suture.

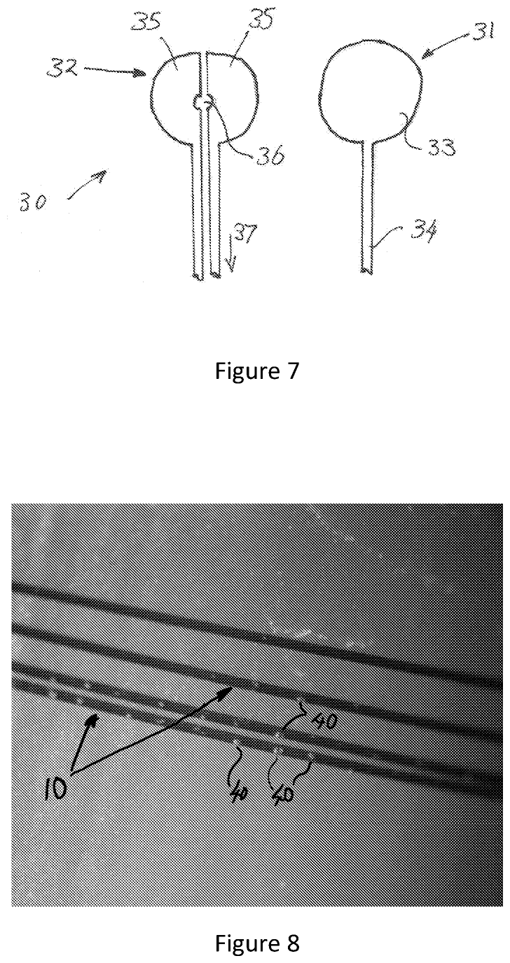

A deformation device 30 is used to deform the distal end 16 to create the distal head 17. In the embodiments described, the deformation device 30 as illustrated in FIG. 7 is a heating device comprising a cautery mechanism 31 and forceps 32 that uses heat to melt the distal end 16. A similar device can be used to create the proximal head 20 prior to surgery, and at the time of manufacturing or modifying the suture.

The cautery mechanism 31 is a disposable or is a sterilisable device that can be attached to a diathermy machine. The cautery mechanism 31 is shaped as a paddle with a concave curved base plate 33 containing a heating element and an arm 34 that attaches to the diathermy machine. The base plate may have a curvature of 7 mm to 8 mm matching the curved surface of the cornea, or the base plate may otherwise be flat.

The forceps make contact with the suture to hold the suture in place so that the base plate 33 of the cautery mechanism can be pressed against the forceps to swage the suture's distal end into a cap. The forceps comprise two crescent-shaped, or semi-elliptical, grasping parts 35 that when brought together by gripping handles 37 create an elliptical flanged head with a central opening 36 through which the suture 10 extends. The flanged shape of the forceps additionally acts as a shield between the cautery mechanism and the cornea to protect the cornea from corneal burn.

The diathermy machine (not shown) will generate a gradual increase in heat proportional to the travel of a foot pedal of the diathermy machine. It generates a controlled temperature (e.g. 40-180 deg C.) to allow melting of the suture. The diathermy machine can be controlled to prevent excessive melting of the suture and thermal burn of the cornea. The heated portion of the paddle does not make direct contact with the eye but only the suture. The application of heat melts the suture tip and the added pressure applied by the surgeon using the heating element base plate will flatten the melted material against the cornea creating a swaged cap to form the distal head 17, which has a size that is approximately 2-3 times larger than the cross-section of the shaft 15. The result will be a suture cap elliptical in shape with its long axis parallel to the corneal limbus. The tear film at the cornea offers immediate cooling. The distal head formed is sufficiently thin to be covered by corneal epithelium when it grows.

The sutures may bear markings to assist the surgeon in selecting the correct suture length for the desired degree of tension. FIG. 8 illustrates several lengths of suture 10 bearing visible markings 40. The markings 40 are generated using a laser to score notches into the surface of the suture at 100 um intervals. A variety of lasers could be used to produce markings including from YAG (Yttrium Aluminum Garnet) lasers to UV (Ultra Violet) lasers. These markings can disturb the smooth surface of the suture which can advantageously increase grip of the suture allowing easier handling. These lasers would not be expected to significantly alter the tensile strength of the suture material.

The distance between the markings need not be equidistant but gap size between markings can increase or decrease in one direction to follow non-linear outcomes and to alter the tension by variable amounts. Referring to the application of corneal transplantation the distances can be linear or geometric or logarithmic and calibrated to proportional astigmatism. The markings can be seen through and outside of the cornea, which is useful for adjusting suture length postoperatively.

The patient will be assessed postoperatively and if the suture is too loose, then again the central region of the proximal head 20 can be pushed with the counter pressure instrument into the cornea making the distal end portion protrude, and exposing the distal head for repeat melting into a new distal head. It may not be necessary to use counter pressure if the surgeon is able to sufficiently grip the distal end with the forceps without the need to push the suture through. The markings on the suture will give an indication of the amount of astigmatic correction that can be achieve proportional to the melting of the suture when adjusting. Furthermore, a guidance chart may be provided to assist surgeons in how far the suture should be adjusted. For example, the chart may show that adjustment by 1 notch is equal to 1 dioppre of astigmatism.

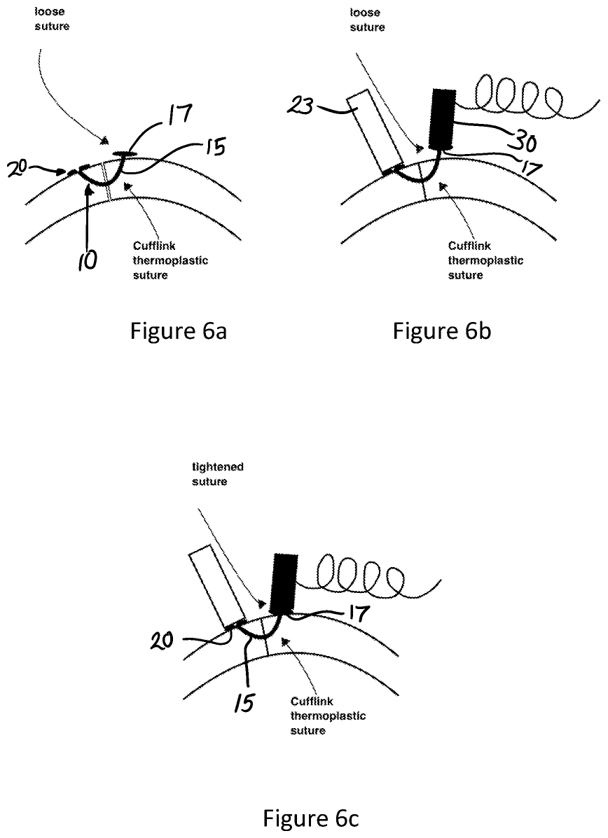

FIGS. 6a to 6c illustrate the adjustment of an implanted suture 10 closing an incision 14. The implanted suture is shown in FIG. 6a being lose, possibly due to de-swelling of the cornea 12, 13. In FIG. 6b counter pressure is applied by way of the counter pressure instrument 23, which makes the distal head 17 protrude further out of the surface of the tissue. The heating element (in the cautery mechanism 31) is applied to the distal head 17 to melt it and shrink the distal head to be flush against the cornea thereby tensioning the implanted suture by shortening its length while implanted.

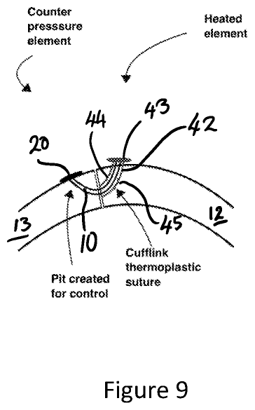

In another variation illustrated in FIG. 9, the suture is threaded through a hollow sleeve 42 having a flanged cap 43 with a central hole (not shown) through which the suture protrudes so that it can be adjusted in length. The sleeve 42 would be inserted on one side of the incision 14 and the suture, as described in earlier embodiments, would be passed through the cornea tissue on the other side of the incision then threaded into the sleeve, out of the cap 43 and the suture needle cut. A similar deformation of the distal end of the suture is then performed using a heating mechanism where the deformed suture is flattened against the sleeve's cap 43.

The suture may have ridges along its length at periodic intervals. The sleeve 42 may also have along its length internal ridges 44 and/or external ridges 45. The suture will thread through the centre to emerge at the cap, the internal ridges 44 will lock against the suture ridges holding the suture because the engaging ridges between the internal surface of the sleeve and the suture act as a ratchet mechanism to allow the suture to be pulled out of the cap 43 but guard against it slipping back in. This would make it easier for a surgeon to deform the distal end of the suture, possibly without the need for forceps.

The sutures can either be cut so a small portion is protruding into the cap or looped and heated to fuse with itself to produce an anchor point. Postoperatively if the suture needs tightening more of the suture could be pulled through the sleeve and cap either directly or by pulling the loop using forceps. Once the surgeon is satisfied with the tension, they could permanently lock the suture with a handheld heating element

The present invention provides improvements over known suturing techniques, particularly where sutures may need to be adjusted postoperatively. The surgical suture as described herein is attractive for use in surgery as it offers a straightforward and efficient stitching technique that can be consistently repeated with similar results. The end stitch is a clean stitch cleanly capped at both ends that sit flush against the tissue thereby mitigating areas for infection and allowing tissue to grow over if desired. Furthermore, the present suture avoids the added time and effort with known sutures of tying knots as only one end of the suture requires finishing and this can be done using a deformation mechanism (the other end being previously preformed with a cap/head).

In the claims which follow and in the preceding description of the invention, except where the context requires otherwise due to express language or necessary implication, the word “comprise” or variations such as “comprises” or “comprising” is used in an inclusive sense, namely, to specify the presence of the stated features but not to preclude the presence or addition of further features in various embodiments of the invention.

It is to be understood that the a foregoing description refers merely to preferred embodiments of invention, and that variations and modifications will be possible thereto without departing from the spirit and scope of the invention, the ambit of which is to be determined from the following claims.

1. A surgical suture comprising:
an elongate body having a proximal head for anchoring the suture;
at least part of the elongate body being deformable to create a distal head, the surgical suture suitable to secure tissue between the proximal head and the distal head.

2. A surgical suture according to claim 1, wherein the elongate body is deformable by at least one of: melting, polymerisation, or moulding with a solvent.

3. A surgical suture according to claim 2, wherein the elongate body is deformable when heated to the melting point of the material of the elongate body.

4. A surgical suture according to claim 1, wherein the elongate body is suitable for insertion through and protruding at least partly out of tissue, the distal head being created from part of the elongate body protruding out of the tissue after insertion.

5. A surgical suture according to claim 1 having suitable stiffness such that force applied to one head is transferred along the elongate body portion to the other head.

6. A surgical suture according to claim 1, wherein the suture is formed from a material having a stiffness of 2.5×10⁹ Pa or greater.

7. A surgical suture according to claim 1, wherein the length of the suture is defined by the length of the elongate body between the proximal head and the distal head, the length of the suture being adjustable after creating the distal head.

8. A surgical suture according to claim 7, wherein the elongate body is made of a deformable material that can be repeatedly cut and deformed to create a new distal head or a new proximal head.

9. A surgical suture according to claim 1, wherein the proximal head is integrally preformed with the elongate body.

10. A surgical suture according to claim 1, wherein the suture comprises a thermoplastic material.

11. (canceled)

12. A surgical suture according to claim 1 wherein the proximal head is a flanged head that has a diameter, the diameter being at least twice the diameter of the elongate body.

13. A surgical suture according to claim 1 wherein the proximal head is tapered out from elongate body to an outer flange perimeter at an angle of approximately 45° to 90°.

14. (canceled)

15. A surgical suture according claim 1, comprising a sharp tip preformed at a distal end of the elongate body to act as a needle.

16. (canceled)

17. A method for inserting the surgical suture of claim 1 to close an incision, comprising the steps of:
inserting the elongate body into a first surface of tissue on a first side of an incision, across the incision and through the tissue to protrude out of the tissue on a second side of an incision until the proximal end anchors the surgical suture at the first surface of the tissue to prevent further insertion;
deforming an exposed part of the elongated body protruding out of the tissue to create a distal head, the surgical suture closing the incision by securing the tissue between the proximal head and the distal head.

18. The method according to claim 17, including adjusting the length of the suture by removing at least one of the proximal head or distal head to create a tip of the elongate body and deforming the tip to create a new head portion, resulting in the elongate body having a shorter length.

19. The method according to claim 17, including deforming the exposed part or tip of the elongate body by heating the exposed part or tip and applying a pressing force to swage the exposed part or tip thereby creating a distal head.

20. The method according to claim 17, including grasping the exposed part or tip using forceps having a flanged head and pressing down onto the exposed part or tip against the flanged forceps head using a heated plate to deform the exposed part or tip into a head.

21. A surgical suture system comprising:
a suture according to claim 1, and a deformation device comprising a cautery mechanism and forceps for holding the elongate body out of the tissue, the cautery mechanism suitable for melting a section of the elongate body portion to create a head portion.

22. A surgical suture system according to claim 21, the cautery mechanism having a flat or curved heated base plate, suitable for applying pressure to and melting the elongate body to create the head portion.

23. A surgical suture system according to claim 21, the forceps comprising semi-elliptical grasping parts having a central opening through which the elongate body extends, and the grasping parts being flanged so as to, in use, shield the tissue from heat generated by the cautery mechanism.
